SEQUENCE LISTING (8 sequences)

SEQ ID NO: 1 - DNA, 339 bp, Artificial Sequence
  DAP12 nucleotide sequence
atggggggacttgaaccctgcagcaggttcctgctcctgcctctcctgctggctgtaagtggtctccgtcctgtccaggtccaggcccagagcgattgcagttgctctacggtgagcccgggcgtgctggcagggatcgtgatgggagacctggtgctgacagtgctcattgccctggccgtgtacttcctgggccggctggtccctcgggggcgaggggctgcggaggcagcgacccggaaacagcgtatcactgagaccgagtcgccttatcaggagctccagggtcagaggtcggatgtctacagcgacctcaacacacagaggccgtattacaaa

SEQ ID NO: 2 - protein, 113 aa, Artificial Sequence
  DAP12 amino acid sequence
MGGLEPCSRFLLLPLLLAVSGLRPVQVQAQSDCSCSTVSPGVLAGIVMGDLVLTVLIALAVYFLGRLVPRGRGAAEAATRKQRITETESPYQELQGQRSDVYSDLNTQRPYYK

SEQ ID NO: 3 - DNA, 57 bp, Artificial Sequence
  T2A nucleotide sequence
ggagagggcagaggaagtcttctaacatgcggtgacgtggaggagaatcccggccct

SEQ ID NO: 4 - protein, 19 aa, Artificial Sequence
  T2A amino acid sequence
GEGRGSLLTCGDVEENPGP

SEQ ID NO: 5 - DNA, 720 bp, Artificial Sequence
  scfv nucleotide sequence
caggtacaactgcagcagtctgggcctgagctggagaagcctggcgcttcagtgaagatatcctgcaaggcttctggttactcattcactggctacaccatgaactgggtgaagcagagccatggaaagagccttgagtggattggacttattactccttacaatggtgcttctagctacaaccagaagttcaggggcaaggccacattaactgtagacaagtcatccagcacagcctacatggacctcctcagtctgacatctgaagactctgcagtctatttctgtgcaagggggggttacgacgggaggggttttgactactggggccaagggaccacggtcaccgtctcctcaggtggaggcggttcaggcggcggtggctctagcggtggtggatcggacatcgagctcactcagtctccagcaatcatgtctgcatctccaggggagaaggtcaccatgacctgcagtgccagctcaagtgtaagttacatgcactggtaccagcagaagtcaggcacctcccccaaaagatggatttatgacacatccaaactggcttctggagtcccaggtcgcttcagtggcagtgggtctggaaactcttactctctcacaatcagcagcgtggaggctgaagatgatgcaacttattactgccagcagtggagtaagcaccctctcacgtacggtgctgggacaaagttggaaatcaaa

SEQ ID NO: 6 - protein, 240 aa, Artificial Sequence
  scfv amino acid sequence
QVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGQGTTVTVSSGGGGSGGGGSSGGGSDIELTQSPAIMSASPGEKVTMTCSASSSVSYMHWYQQKSGTSPKRWIYDTSKLASGVPGRFSGSGSGNSYSLTISSVEAEDDATYYCQQWSKHPLTYGAGTKLEIK

SEQ ID NO: 7 - DNA, 243 bp, Artificial Sequence
  TREM1cut nucleotide sequence
cctcctaccaccactaaggccttgtgcccactctataccagccccagaactgtgacccaagctccacccaagtcaactgccgatgtctccactcctgactctgaaatcaaccttacaaatgtgacagatatcatcagggttccggtgttcaacattgtcattctcctggctggtggattcctgagtaagagcctggtcttctctgtcctgtttgctgtcacgctgaggtcatttgtaccctag

SEQ ID NO: 8 - protein, 80 aa, Artificial Sequence
  TREM1cut amino acid sequence
PPTTTKALCPLYTSPRTVTQAPPKSTADVSTPDSEINLTNVTDIIRVPVFNIVILLAGGFLSKSLVFSVLFAVTLRSFVP

(19) United States
(12) Patent Application Publication
(10) Pub. No.: US 2022/0280565 A1
(43) Pub. Date: Sep. 8, 2022

(54) Chimeric antigen receptor carrying truncated or untruncated myeloid cell triggering receptor signaling structure and applications thereof

(71) Applicant: NANJING CART MEDICAL TECHNOLOGY LTD., Nanjing, Jiangsu (CN)

(72) Inventors: Enxiu Wang, Nanjing, Jiangsu (CN); Chen Wang, Nanjing, Jiangsu (CN); Hai Zhang, Nanjing, Jiangsu (CN); Guoying Wu, Nanjing, Jiangsu (CN)

(21) Appl. No.: 17/285,084

(22) PCT Filed: May 23, 2019
(86) PCT No.: PCT/CN2019/088050

§ 371 Date: Apr. 13, 2021

(30) Foreign Application Priority Data
Jun. 12, 2018 (CN) 201810600083.6

(51) Int. Cl. A61K 35/17; A61P 35/00; C07K 14/725

(52) U.S. Cl. CPC A61K 35/17; A61P 35/00; C07K 14/7051; A61K 38/00

(57) ABSTRACT

A chimeric antigen receptor (CAR) includes: an antigen-binding domain (scfv) and a signaling domain, wherein the signaling domain includes a first conducting domain and a second conducting domain; the antigen-binding domain is connected between the first conducting domain and the second conducting domain.

### BACKGROUND OF THE PRESENT INVENTION

### Field of Invention

The present invention relates to a technical field of tumor immunotherapy, and more particularly to a chimeric antigen receptor carrying a truncated or untruncated myeloid cell triggering receptor signaling structure and application thereof.

### Description of Related Arts

Chimeric antigen receptor (CAR) is the core component of CAR-T. Using the characteristics of the ligand binding domain, CAR can redirect the specificity and reactivity of selected immune cells, thus conferring T cells an HLA-independent manner to recognize tumor antigens, which allows CAR-engineered T cells to recognize a wider range of targets than the native T-cell surface receptor TCR does. The basic design of CAR includes a tumor-associated antigen (TAA) binding region (usually derived from the scFV segment of the monoclonal antibody antigen binding region), an extracellular hinge region, a transmembrane region, and an intracellular signal area.

Conventional CAR-T is effective for hematological tumors, but is insufficient for solid tumors, which limits its clinical application. From the perspective of safety, cytokine release syndrome (CRS) is a common complication of CAR-T cell therapy and can even be life-threatening. After being infused into the body, CAR-T cells are activated and begin to proliferate because the chimeric antigen receptor specifically binds to the corresponding tumor-associated antigen, which trigger the release of cytokine cascades and mediating multiple types of immune responses, leading to clinical manifestations such as fever, hypotension, dyspnea, coagulopathy, and terminal organ disorders, that is, CRS. The degree of the conventional CAR-T directly affected by the antigen-stimulated secretion of cytokines determines the degree of severity of CRS. From the perspective of effectiveness, the tumor matrix composed of solid cancer-associated fibroblasts (CAFs) provides a physical barrier for CAR-T cell infiltration. CAFs also secrete extracellular matrix proteins to transport T cells from cancer cells. Second, the metabolic microenvironment of the solid tumor is not conducive to the persistence of conventional CAR-T cells, because once tumor formation is activated, tumor cells stop producing ATP through oxidative phosphorylation and stop converting to aerobic glycolysis. As a result, the tumor environment becomes acidic, which is the so-called “Wattock effect”, wherein the pH will drop from 7.4 to 6.5. Finally, hypoxic state of the tumor microenvironment further produces immunosuppression. The tumor cells will produce HIF1-α molecules in a hypoxic environment, which weakens the anti-tumor function of the conventional CAR-T cells by attracting regulatory T cells (Tregs) to the tumor site. Since Tregs suppress the immune response, the therapeutic effect of the conventional CAR-T on solid tumors is limited.

### SUMMARY OF THE PRESENT INVENTION

An object of the present invention is to provide a chimeric antigen receptor (CAR) carrying a myeloid cell triggering receptor TREM1 or TREM2 signaling structure with better safety and curative effect.

The second object of the present invention is to provide an application of the chimeric antigen receptor.

The third object of the present invention is to provide a signaling domain of the chimeric antigen receptor.

The fourth object of the present invention is to provide an application of the signaling domain.

Accordingly, in order to accomplish the above objects, the present invention provides:

a chimeric antigen receptor (CAR), comprising: an antigen-binding domain (scfv) and a signaling domain, wherein the signaling domain comprises a first conducting domain and a second conducting domain; the antigen-binding domain is connected between the first conducting domain and the second conducting domain; the first conducting domain is truncated or untruncated TREM1 or TREM2.

In the chimeric antigen receptor of the present invention, the first conducting domain, the antigen-binding domain, and the second conducting domain in tandem are converted into a multi-chain form capable of transmitting activation signals after the antigen-binding domain specifically binds antigens, and then transmit the activation signals to immune cells for immunotherapy.

The chimeric antigen receptor has a multi-chain structure, which uses the first conducting domain and the second conducting domain to form the signaling domain of the CAR. The antigen-binding domain can specifically bind to a target and induce activation of immune cells, so as to produce an immune response.

The second conducting domain is DAP12, and is connected in tandem with the antigen-binding domain through T2A.

The DAP12 of the present invention is a transmembrane domain, which is widely present on surfaces of natural killer cells, granulocytes, monocytes/macrophages, and is used to transmit the activation signals. The DAP12 has a nucleotide sequence of SEQ ID NO.1 and an amino acid sequence of SEQ ID NO.2.

The T2A of the present invention is used to tandem the second conducting domain and the antigen-binding domain. The T2A has a nucleotide sequence of SEQ ID NO.3 and an amino acid sequence of SEQ ID NO.4.

The first conducting domain of the present invention may be the truncated or untruncated TREM1 or TREM2, wherein a full-length TREM1 gene has a nucleotide sequence as shown in NCBI, GenBank: NM_018643.4, and an amino acid sequence as shown in NCBI, GenBank: NP_061113.1; a full-length TREM2 gene has a nucleotide sequence as shown in NCBI, Accession: NM_018965.3, and an amino acid sequence as shown in NCBI, Accession: NP_061838.1. The truncated TREM1 or TREM2 represents an amino acid sequence of a C-terminus of the full-length amino acid sequence.

In order to improve safety and efficacy of the chimeric antigen receptor, the present invention also provides a preferred first conducting domain, which is the truncated TREM1 amino acid sequence named TREM1_{cut}. The TREM1_{cut} of the present invention is a polypeptide having 40-90 amino acids of a C-terminus of a full-length TREM1 amino acid sequence, preferably a polypeptide having 50-85 amino acids of the C-terminus of the full-length TREM1 amino acid sequence, and more preferably a polypeptide having 60-80 amino acids of the C-terminus of the full-length TREM1 amino acid sequence; or an amino acid sequence having at least 80% identity with the polypeptide, or an amino acid sequence having at least 85% identity with the polypeptide, or an amino acid sequence having at least 90% identity with the polypeptide, or an amino acid sequence having at least 95% identity with the polypeptide.

The present invention also provides a preferred first conducting domain being a polypeptide having 80 amino acids of the C-terminus of the full-length TREM1 amino acid sequence, which has an amino acid sequence of SEQ ID NO.8 and a nucleotide sequence of SEQ ID NO.7.

The antigen-binding domain of the present invention can be commonly selected in the art according to different tumor targets.

Specifically, the chimeric antigen receptor of the present invention is formed by connecting DAP12, T2A, the antigen-binding domain, and the first conducting domain in tandem through 2-10 arbitrary amino acids. According to the present invention, sequence and number of the 2-10 arbitrary amino acids have no significant effect on efficacy of the chimeric antigen receptor, and may be 2-10 of any amino acid sequences.

The chimeric antigen receptor of the present invention may use, for example, a retroviral vector to transfer a nucleic acid encoding the chimeric antigen receptor to an immune cell such as a T cell. When the chimeric antigen receptor binds a target antigen, the first conducting domain and the antigen-binding domain are separated to generate the activation signal which is then transmitted to the immune cells for expressing.

The present invention also provides an immune cell having the above chimeric antigen receptor.

The present invention also provides a tumor immunotherapy method, comprising using the above chimeric antigen receptor or the above immune cell.

The present invention also provides a signaling domain, comprising a first conducting domain and a second conducting domain, wherein the first conducting domain is truncated or untruncated TREM1 or TREM2.

The second conducting domain of the present invention is DAP12, which is a transmembrane domain, which is widely present on surfaces of natural killer cells, granulocytes, monocytes/macrophages, and is used to transmit the activation signals. The DAP12 has a nucleotide sequence of SEQ ID NO.1 and an amino acid sequence of SEQ ID NO.2.

The first conducting domain of the present invention may be the truncated or untruncated TREM1 or TREM2, wherein a full-length TREM1 gene has a nucleotide sequence as shown in NCBI, GenBank: NM_018643.4, and an amino acid sequence as shown in NCBI, GenBank: NP_061113.1; a full-length TREM2 gene has a nucleotide sequence as shown in NCBI, Accession: NM_018965.3, and an amino acid sequence as shown in NCBI, Accession: NP_061838.1. The truncated TREM1 or TREM2 represents an amino acid sequence of a C-terminus of the full-length amino acid sequence.

In order to improve safety and efficacy of the chimeric antigen receptor formed by the signaling domain, the present invention also provides a preferred first conducting domain, which is the truncated TREM1 amino acid sequence named TREM1_{cut}. The TREM1_{cut} of the present invention is a polypeptide having 40-90 amino acids of a C-terminus of a full-length TREM1 amino acid sequence, preferably a polypeptide having 50-85 amino acids of the C-terminus of the full-length TREM1 amino acid sequence, and more preferably a polypeptide having 60-80 amino acids of the C-terminus of the full-length TREM1 amino acid sequence; or an amino acid sequence having at least 80% identity with the polypeptide, or an amino acid sequence having at least 85% identity with the polypeptide, or an amino acid sequence having at least 90% identity with the polypeptide, or an amino acid sequence having at least 95% identity with the polypeptide.

The present invention also provides a preferred first conducting domain being a polypeptide having 80 amino acids of the C-terminus of the full-length TREM1 amino acid sequence, which has an amino acid sequence of SEQ ID NO.8 and a nucleotide sequence of SEQ ID NO.7.

According to the signaling domain of the present invention, the transmembrane receptor DAP12 is combined with the first conducting domain to form the signaling domain of the CAR. When the CAR specifically binds to a ligand in the target, it can induce the activation of the immune cells, so as to produce the immune response.

The present invention provides a method for preparation of a chimeric antigen receptor or tumor immunotherapy, comprising using the above signaling domain.

According to the present invention, the C-terminus refers to a polypeptide that is truncated from the first amino acid in a C segment. For example, a polypeptide of 40-90 amino acids of the C segment indicates a polypeptide from the first amino acid in the C segment to any of the 40-90 amino acids.

Beneficial effects of the present invention:

(1) When the CAR structure of the present invention is stimulated by an antigen, a secreted cytokine level is extremely low, which can ensure the safety of clinical application, which means the safety of clinical application is higher.

(2) The CAR structure of the present invention has proved its significant effect on solid tumors through in vitro functional experiments. Therefore, the present invention can not only be applied to the treatment of blood tumors, but also expand the application of CAR-T in the treatment of solid tumors.

(3) The CAR structure of the present invention has stronger in vitro antigen-positive tumor cell killing ability and better anti-tumor activity.

### BRIEF DESCRIPTION OF THE DRAWINGS

FIG. 1 illustrates lentiviral vectors containing different signaling domains;

FIG. 2 illustrates a positive expression rate of a MSLN antigen CAR structure on surfaces of T cells recognized by a flow cytometry after 7 days of lentivirus infection of MSLN4CAR-T cells;

FIG. 3 illustrates proliferation of CAR-T cells after the lentivirus infection;

FIG. 4 illustrates IL-2 secretion of the MSLN4CAR-T cells under antigen stimulation;

FIG. 5 illustrates IFN-γ secretion of the MSLN4CAR-T cells under the antigen stimulation;

FIG. 6 illustrates IL-2 and IFN-γ secretion of CAR-T in different groups under the antigen stimulation;

FIG. 7 illustrates killing effects of the CAR-T in different groups on antigen-positive tumor cell lines.

### DETAILED DESCRIPTION OF THE PREFERRED EMBODIMENT

Embodiments of the present invention will be described in detail below with the accompanying drawings. Experimental methods without specific conditions in the embodiments are generally based on common conditions, such as those described in the Molecular Cloning Experiment Guide (Third Edition, J. Sambrook et al.) or those recommended by the manufacturer. Unless otherwise specified, test materials used in the following embodiments are commercially available.

### EMBODIMENT 1

### Construction of CAR Lentivirus Containing DAP12-T2A-scFv-TREM1_{cut}

In order to prove that CAR-T cells containing DAP12-TREM1_{cut} intracellular signal domain have more advantages than conventional CAR-T cells containing 4-1BB-CD3ζ, DAP12-KIRS2 and single DAP12 stimulation signals, it is necessary to separately construct viral vectors with different combinations of stimulation signals. In the embodiment 1, a single-chain antibody targeting human mesothelin (MSLN) is used as a unified extracellular antigen recognizing structure, wherein the following five chimeric antigen receptors need to be constructed (shown in FIG. 1):

MSLN (scfv)-CD8α-4-1BB-CD3ζ (MSLN1)

DAP12-T2A-MSLN (scfv) (MSLN2)

DAP12-T2A-MSLN (scfv)-KIRS2 (MSLN3)

DAP12-T2A-MSLN (scfv)-TREM1_{cut} (MSLN4)

DAP12-T2A-MSLN (scfv)-TREM1_{wt} (MSLN5)

1. Synthesis of Human Mesothelin-Targeting Chimeric Antigen Receptor Gene Sequences Containing Different Intracellular Stimulation Signals

Natural killer activated receptor (DAP12), T2A, single chain antibody scfv (MSLN (scfv)) against human mesothelin, myeloid cell triggering receptor (TREM1_{wt}), and truncated myeloid cell triggering receptor (TREM1_{cut}) are synthesized, whose structures are shown in FIG. 1. The DAP12 has a nucleotide sequence of SEQ ID NO.1 and an amino acid sequence of SEQ ID NO.2. The T2A has a nucleotide sequence of SEQ ID NO.3 and an amino acid sequence of SEQ ID NO.4. The single-chain antibody (MSLN) scfv against human mesothelin has a nucleotide sequence of SEQ ID NO.5 and an amino acid sequence of SEQ ID NO.6. A full-length TREM1 gene has a nucleotide sequence as shown in NCBI, GenBank: AJ225109.1 and an amino acid sequence as shown in NCBI, GenBank: CAB39168.1. The TREM1_{cut} has a nucleotide sequence of SEQ ID NO.7 and an amino acid sequence of SEQ ID NO.8. KIRS2 has nucleotide and amino acid sequences as shown in Chinese patent (Targeted cytotoxic cells with chimeric receptors for adoptive immunotherapy, publication number: CN107580628 A). CD8α-4-1BB-CD3ζ has nucleotide and amino acid sequences as shown in U.S. patent (Methods for treatment of cancer, US 20130309258A1).

2. Construction of Lentiviral Vector Expressing Chimeric Antigen Receptor

pELNS Dap12-T2A-MSLN-KIRS2 is kept by Nanjing Kati Medical Technology Co., Ltd., or constructed according to literature (Enxiu Wang et al. Generation of Potent T-cell Immunotherapy for Cancer Using DAP12-Based, Multichain, Chimeric Immunoreceptors. 2015, Cancer Immunology Research, 3 (7): 815). The truncated TREM1_{cut} gene is synthesized by Nanjing Kingsray Biotechnology Company and pUC19-TREM1_{cut} plasmid is provided. The plasmids pELNS Dap12-T2A-MSLN-KIRS2 and pUC19-TREM1_{cut} is double-digested by NheI and SalI (purchased from Takara), wherein digestion reaction is performed according to instructions to obtain a DNA fragment with a pELNS Dap12-T2A-MSLN fragment of about 8900 bp and a truncated TREM1_{cut} fragment of about 243 bp. A recovery kit (from Takara) is used for DNA fragment recovery as described in the specification, thereby recovering obtained pELNS Dap12-T2A-MSLN and TREM1_{cut} genes. Then the target fragment TREM1_{cut} and the vector fragment pELNS Dap12-T2A-MSLN are connected through T4 ligase (purchased from Takara) to obtain a lentiviral vector expressing a chimeric antigen receptor, named pELNS Dap12-T2A-MSLN-TREM1_{cut} (MSLN4 for short). 5 μL of the lentiviral vector MSLN4 is transformed into E. coli TOP10 competent cells (purchased from Nanjing Anjieyou Biotechnology Co., Ltd.). After culturing at 37° C. for 16 hours, monoclonal antibodies are picked. Then the picked monoclonal antibodies are cultured at 37° C. for 12 hours before plasmids are extracted with a plasmid extraction kit (purchased from Takara) as described in the specification.

According to the above methods, pELNS MSLN-CD8α-4-1BB-CD3ζ (MSLN1 for short); pELNS Dap12-T2A-MSLN (MSLN2 for short); pELNS Dap12-T2A-MSLN-KIRS2 (MSLN3 for short); pELNS Dap12-T2A-MSLN-TREM1_{cut} (MSLN4 for short); pELNS Dap12-T2A-MSLN-TREM1_{wt} (MSLNS for short) lentiviral vectors are also constructed.

### 3. Lentivirus Packaging

According to embodiment 1, the lentivirus is packaged by a calcium phosphate method comprising specific steps of:

(1) passaging 293T cells the next day

seeding 5×10⁶ cells in each T150 cell flask, wherein after 48 hours, the number of cells should reach 20-25 million/flask;

(2) laying the 293T cells in bottles

a) taking one T150 cell flask as an example, gently washing the cells twice with about 15 ml of 1×PBS;

b) adding 3 ml 0.25% trypsin-2.21mM EDTA

c) waiting until the cells fall off, adding 12 ml 10% (wt) FBS (purchased from Gibico) DMEM medium (purchased from corning) to the cells that have fallen off;

d) collecting and transferring the cells to a sterile centrifuge tube, centrifuging at 1000 rpm for 10 minutes;

e) removing supernatant and resuspending pellet in 10 ml 10% (wt) FBS DMEM medium;

f) counting the cells, calculating a volume required for 12×10⁶ cells based on a cell concentration; and

g) combining the cells with 25 ml 10% (wt) FBS DMEM medium, putting into a T150 cell flask, and shaking gently to evenly distribute the cells to a bottom of the cell flask; culturing overnight at 37° C. in a 5% CO₂ incubator;

(3) performing cell transfection

observing the cells, and starting transfection when a cell density is about 80%-90%

a) gently removing the culture medium 30-60 minutes before transfection;

b) mixing plasmid DNA and calcium chloride solution, wherein taking one T150 bottle as an example, 28 ug pRSV.rev (purchased from Invitrogen), 28 ug pGAG-Pol (purchased from Invitrogen), 11 ug pVSVG (purchased from Invitrogen), and 23 ug recombinant lentivirus expression plasmid pELNS Dap12-T2A-MSLN-TREM1_{cut} are added to 1.5 ml calcium chloride solution and mixed;

c) adding 1.5 ml BBS solution to a 15 ml sterile centrifuge tube, fully mixing the DNA-calcium chloride solution with a 1 ml pipette tip, and then adding dropwise to the BBS solution; quickly mixing 15-20 times, and incubating at room temperature for 25-30 minutes.

d) using a 5 ml pipette to add the DNA-calcium chloride-BBS mixture (purchased from Shanghai Biyuntian Biotechnology Co., Ltd.) evenly and dropwise to the T150 bottle, wherein the cells are cultured in a 37° C. cell incubator containing 5% carbon dioxide, and medium is changed after 6 hours; and

e) after changing the medium after 6 hours, and gently shaking a culture plate several times to fully suspend some calcium phosphate precipitates; removing the medium containing the calcium phosphate precipitates, adding 20 ml fresh 5% (wt) FBS DMEM medium, and continuing the culture;

(4) collecting virus supernatant for the first time

a) collecting 293T cell culture supernatant transfected the previous day into a centrifuge tube, centrifuging at 1000rpm for 5 minutes, labeling, and temporarily storing in a refrigerator at 4° C.; and

b) adding pre-warmed 20 ml 5% (wt) FBS DMEM medium to a cell flask, and incubating the cells at 37° C. overnight;

(5) collecting the virus supernatant for the second time (48 h/day 4)

(6) filtering the supernatant

mixing two collected supernatants together and filtering through a 0.45 μm filter to remove cell debris

(7) performing virus concentration

centrifuging overnight at 4° C. and 12000-24000 rpm

(8) storing virus

after centrifugation, removing all supernatant, and adding fresh 5% (wt) FBS DMEM medium to resuspend; aliquoting the virus, and quickly storing in a −80° C. refrigerator for future use; and (9) determining lentivirus titer

a) infecting 293T cells with the virus

plating 293T cells into a 24-well plate before infection, and adding 200 μL purified concentrated virus to the 293T cells; after 24 hours, replacing the medium with 10% (wt) FBS DMEM medium; after 72 hours of infection, centrifuging at 1200 r/min for 5 min to collect the cells and extract genome;

b) extracting the genome

wherein a genomic extraction kit is purchased from Takara and is operated according to kit instructions; and

c) performing qPCR for virus titer

wherein a reaction system is as follows: Probe qPCR Mix 12.5 μL (purchased from Takara), upstream primer 0.5 μL (synthesized by Nanjing Kingsray), downstream primer 0.5 μL (synthesized by Nanjing Kingsray), probe 1 μL (synthesized by Nanjing Kingsray, template 2 μL, sterilized water 8.5 μL; the reaction system is 25 μL and reaction conditions are set according to instructions; after reaction, data are analyzed by analysis software, and the virus titer is calculated according to a standard curve, wherein calculation result shows that the virus titer is 1.3×10⁶ TU/ml.

### EMBODIMENT 2

### Virus Infection of T Cells

1. Isolation and Activation of T cells and Virus Infection

(1) Isolation of Human Peripheral Blood Mononuclear Cells

About 10 ml peripheral blood are collected with an anticoagulant blood collection tube, and naturally settled at room temperature (18-25° C.) for about 30 min. Upper plasma are collected, centrifuged at 5000 r/min for 10 min, and added to lymphocyte separation solution (purchased from Tianjin Ouyang Biological Products Technology Co., Ltd.) with a volume ratio of 1:1 before gradient centrifugation at 3000 r/min for 30 min. After centrifugation, layers are separated in the centrifuge tube from top to bottom, wherein the first layer is a plasma layer, the second layer is a lymphocyte albuginea layer, the third layer is a transparent separating liquid layer, and the fourth layer is a red blood cell layer. The lymphocyte albuginea layer is aspirated, washed twice with PBS, and centrifuged twice at 1500 r/min for 10 min. The cells are resuspended in PBS. The human peripheral blood mononuclear cells are cultured with 5% autologous plasma+300 IU/ml recombinant human IL-2+KBM581 complete medium.

(2) Infecting T Lymphocytes by Lentivirus

Newly prepared mononuclear cells PBMC are cultured in the 5% autologous plasma+300 IU/ml recombinant human IL-2+KBM581 complete medium, wherein IL-2 is purchased from R&D Systems, KBM581 is purchased from Corning. CD3/CD28 Dynabeads (purchased from invitrogen) are added on day 0 to activate the T cells. Lentivirus infection is performed in the first 3 days. Lentiviral vectors corresponding to 0.25 MOI are added. Uninfected T lymphocytes are used as blank controls. After 48 hours, the medium was replaced with 5% autologous plasma+300 IU/ml recombinant human IL-2+KBM581 complete medium and the culture is continued for 7-9 days.

2. Detection of CAR Positive Rate in the T Cells

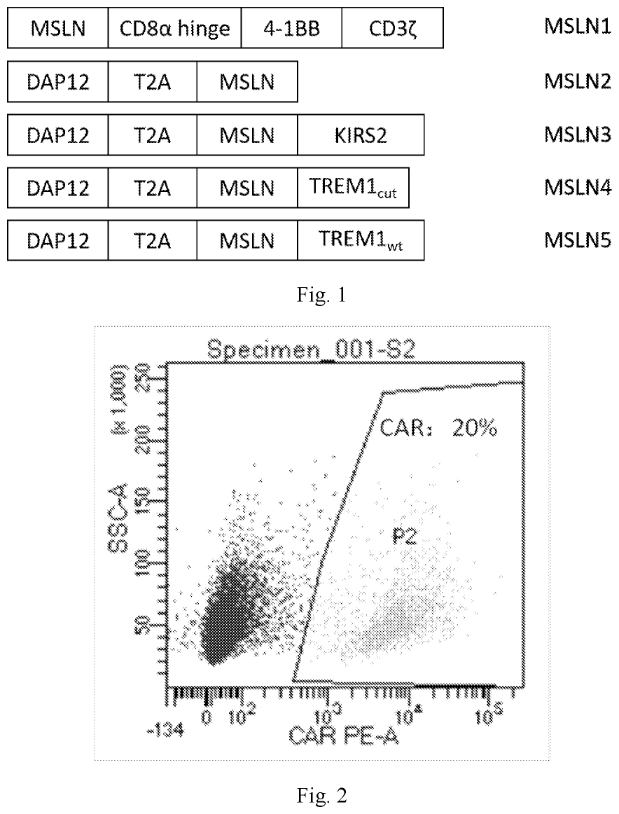

Virus-infected T cells cultured to day 7 are centrifuged at 1200 r/min for 5 min. Supernatant is discarded to collect the cells, and the cells are resuspended with a PBS solution containing 1% FBS in volume fraction. A density of the cells is adjusted to 1×10⁵ cells/ml, and biotin-labeled goat anti-mouse F(ab)₂ (Jackson ImmunoResearch) is added. Then Streptavidin-PE (BD Biosciences) is added before incubating for 15 min at 4° C. and washing twice with PBS solution. A flow cytometry is used for detecting, which shows that after 7 days of culture, CAR positive rate of the CAR-T cell is 41% in the MSLN1 virus infection group, 52% in the MSLN2 virus infection group, 59% in the MSLN3 virus infection group, 20% in the MSLN4 virus infection group (see FIG. 2), and 49% in the MSLN5 virus infection group.

### EMBODIMENT 3

### Effect of Virus-Infected CAR-T Cells on Cell Proliferation

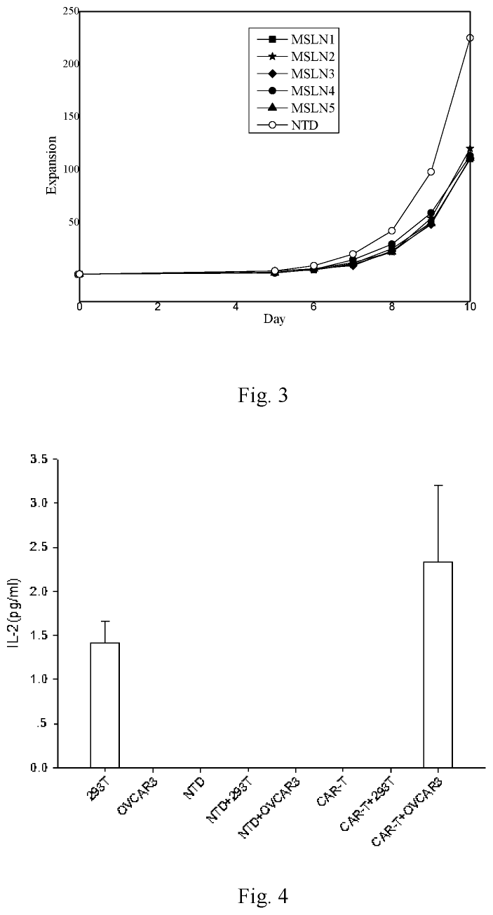

After the T cells in each group are infected by the virus, the T cells are counted every 1-2 days with the 5% autologous plasma+300 IU/ml recombinant human IL-2+KBM581 complete medium. Then growth of T lymphocytes is observed and and results are shown in FIG. 3, which indicates that the cells infected with CAR-expressing virus can still form typical proliferating cloning groups. By counting the cells and plotting cell proliferation curves, it can be seen that proliferation of infected MSLN4CAR-T cells is similar to those of MSLN1, MSLN2, MSLN3, and MSLN5 CAR-T. The proliferation is slightly weaker than that of non-infected T cells (NTD in FIG. 3).

### EMBODIMENT 4

### Detection of Cytokine Secretion of Virus-Infected CAR-T Cells

(1) using Elisa's method for cytokine detection with kits from R&D;

(2) diluting a standard: preparing and serially numbering seven 1 ml centrifuge tubes; adding 500 μL standard dilution to each centrifuge tube, and then adding 500 μL original standard to one of the numbered centrifuge tube and mixing thoroughly; adding 500 μL mixture of the numbered centrifuge tube to a second centrifuge tube and mixing thoroughly; adding 500 μL mixture of the second centrifuge tube to a third centrifuge tube and mixing thoroughly; adding 500 μL mixture of the third centrifuge tube to a fourth centrifuge tube and mixing thoroughly; adding 500 μL mixture of the fourth centrifuge tube to a fifth centrifuge tube and mixing thoroughly; adding 500 μL mixture of the fifth centrifuge tube to a sixth centrifuge tube and mixing thoroughly; adding 500 μL mixture of the sixth centrifuge tube to a seventh centrifuge tube and mixing thoroughly;

(3) setting standard wells on an enzyme-labeled coating plate, and adding 100 μL standards of different concentrations in sequence, with 2-3 parallel wells of each concentration;

(4) loading sample: setting blank wells (wherein blank control wells are replaced with water, and enzyme-labeled reagents and biotin-labeled antibodies are handled as usual) and sample wells; adding 100 μL samples to the sample wells on the enzyme-labeled coating plate, wherein the samples are added to well bottoms of the enzyme-labeled coating plate while avoiding contacting well walls; and shaking gently to mix;

(5) incubating: incubating at room temperature for 2 h;

(6) washing: discarding liquid and spinning to dry; then adding 200 μL washing solution to each well and discarding after 30 seconds; repeating 3 times and patting to dry;

(7) adding antibody: adding 100 μL detection antibody to the enzyme-labeled coating plate;

(8) incubating: same operation as the step (5);

(9) washing: same operation as the step (6);

(10) labeling: adding 100 μL horseradish peroxidase-labeled streptavidin to each well;

(11) incubating: incubating at room temperature for 20 min in the dark;

(12) washing: same operation as the step (6);

(13) performing color development: adding 100 μL color development solution to each well, shaking gently to mix, and incubating at room temperature for 20 min in the dark;

(14) stopping: adding 50 μL stop solution to each well to stop the reaction;

and (15) measuring: zeroing with a blank value and sequentially measuring absorbance (OD value) of each well at 450 nm, wherein measurement should be performed within 15 min after adding the stop solution.

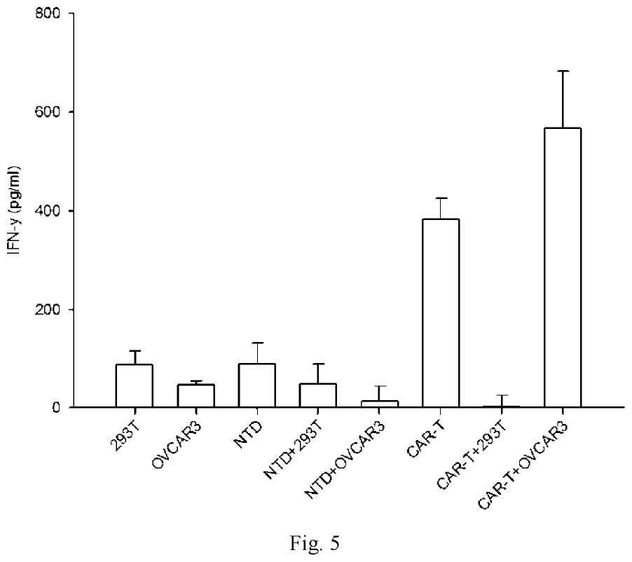

Target cells with different antigen expression levels are selected to be co-cultured with MSLN4 CAR-T, so as to detect secretion levels of IL-2 and IFN-γ of MSLN4 CAR-T in response to antigen stimulation, wherein OVCAR3 (MSLN high expression) and 293T (MSLN negative) are selected as the target cells to show that MSLN4 CAR-T specifically secretes IL-2 and IFN-γ when stimulated by MSLN antigen. Results reflect that MSLN4 CAR has different response effects on the target cells with different antigen expression levels, wherein CAR-T of the MSLN4 significantly secretes IFN-γ and IL-2 when co-cultured with MSLN high expression target cells OVCAR3 (see FIGS. 4 and 5), indicating that MSLN4 CAR has a response to antigen-positive tumor cells.

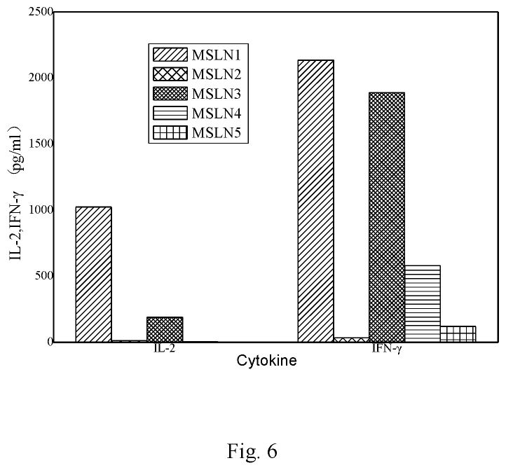

On the other hand, the secretion levels of IL-2 and IFN-γ cytokines in each group are compared when MSLN1, MSLN2, MSLN3, MSLN4, MSLN5 CAR-T are co-cultured with OVCAR3. Results are shown in FIG. 6. The secretion level of the MSLN4 CAR-T is significantly higher than those of MSLN2 and MSLN5 CAR-T groups (since an antitumor effect of MSLN2 CAR-T is very weak, there is no secretion of cytokines), and is lower than those of MSLN1 and MSLN3. In vitro cytokine secretion results show that the MSLN4 CAR-T and the MSLN5 CAR-T produce lower levels of cytokines, implicating improvement of clinical application safety.

### EMBODIMENT 5

### Evaluation of Killing Effect of Virus-Infected CAR-T Cells In Vitro

(1) separately culturing the target cells comprising OVCAR3 cells (MSLN high expression cell line), 293T (MSLN negative cell line), and effector cells comprising MSLN1, MSLN2, MSLN3, MSLN4, and MSLN5 CAR-T cells;

(2) collecting the target cells and the effector cells, centrifuging at 1500 rpm for 5 min, and discarding supernatant;

(3) resuspending the target cells and the effector cells with 10% FBS+1640 complete medium;

(4) using a real-time cell analysis system (RTCA) to add 50 μL 1640 medium in wells of E-Plate16;

(5) using the RTCA to detect baseline and confirming that the selected wells are in normal contact;

(6) setting effect target ratios to 0:1, 1:1, 5:1, and 10:1;

(7) taking out the E-Plate16 and adding 100 μL uniformly mixed target cell suspension into each well according to the effect target ratios, wherein there are 10⁴ cells/100 μL in each well;

(8) putting the E-Plate16 in an incubator overnight at 37° C. and 5% CO₂;

(9) on the second day, removing the E-Plate16, adding 50 μL corresponding effector cells, and calculating a killing rate after 8 hours of adding the effector cells; and

$$Killing Efficiency = \frac{CI ( No Effector ) - CI ( Effector )}{CI ( No Effector )} \times 100 \% . \qquad ( 10 )$$

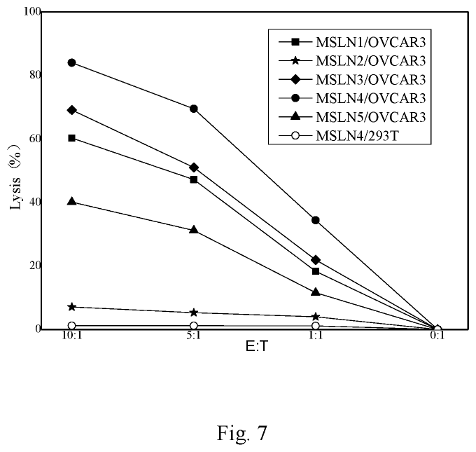

Test results are shown in FIG. 7. The killing effect of CAR-T of MSLN4 on MSLN antigen-positive tumor cells is significantly higher than those of the other four groups, especially than those of the MSLN2 and the MSLN5 groups. Referring to FIGS. 6 and 7, results of in vitro killing experiments indicate that truncated myeloid cell triggering receptors, namely TREM1_{cut} as the first signaling domain, has strong antitumor activity on the basis of improving CAR safety. The design of CAR signal region is conducive to clinical application.

Last but not least, the above embodiments are only used to illustrate the technical solutions of the present invention and are not limiting. Although the present invention has been described in detail through the above embodiments, those skilled in the art should understand that various modifications can be made without departing from the scope defined by the claims of the present invention.

1. A chimeric antigen receptor, comprising: an antigen-binding domain and a signaling domain, wherein the signaling domain comprises a first conducting domain and a second conducting domain; the antigen-binding domain is connected between the first conducting domain and the second conducting domain; the first conducting domain is truncated or untruncated TREM1 or TREM2.

2. The chimeric antigen receptor, as recited in claim 1, wherein the first conducting domain, the antigen-binding domain, and the second conducting domain in tandem are converted into a multi-chain form capable of transmitting activation signals after the antigen-binding domain specifically binds antigens, and then transmit the activation signals to immune cells for immunotherapy.

3. The chimeric antigen receptor, as recited in claim 1, wherein the second conducting domain is DAP12, and is connected in tandem with the antigen-binding domain through T2A; the DAP12 has a nucleotide sequence of SEQ ID NO.1 and an amino acid sequence of SEQ ID NO.2; the T2A has a nucleotide sequence of SEQ ID NO.3 and an amino acid sequence of SEQ ID NO.4; the first conducting domain is a TREM1 amino acid sequence, which is a polypeptide having 40-90 amino acids of a C-terminus of a full-length TREM1 amino acid sequence, or a polypeptide having 50-85 amino acids of the C-terminus of the full-length TREM1 amino acid sequence, or a polypeptide having 60-80 amino acids of the C-terminus of the full-length TREM1 amino acid sequence; or a nucleotide sequence having at least 80% identity with the polypeptide, or a nucleotide sequence having at least 85% identity with the polypeptide, or a nucleotide sequence having at least 90% identity with the polypeptide, or a nucleotide sequence having at least 95% identity with the polypeptide.

4. The chimeric antigen receptor, as recited in claim 3, wherein the first conducting domain has an amino acid sequence of SEQ ID NO.8 and a nucleotide sequence of SEQ ID NO.7.

5. The chimeric antigen receptor, as recited in claim 1, wherein the chimeric antigen receptor is formed by connecting DAP12, T2A, the antigen-binding domain, and the first conducting domain in tandem through 2-10 arbitrary amino acids.

6. An immune cell having the chimeric antigen receptor as recited in claim 1.

7. A tumor immunotherapy method, comprising using the chimeric antigen receptor as recited in claim 1.

8. A signaling domain, comprising a first conducting domain and a second conducting domain, wherein the first conducting domain is truncated or untruncated TREM1 or TREM2.

9. The signaling domain, as recited in claim 8, wherein the second conducting domain is DAP12; the DAP12 has a nucleotide sequence of SEQ ID NO.1 and an amino acid sequence of SEQ ID NO.2; the first conducting domain is a truncated TREM1 amino acid sequence, which is a polypeptide having 50-80 amino acids of a C-terminus of a full-length TREM1 amino acid sequence, or a polypeptide having 60-80 amino acids of the C-terminus of the full-length TREM1 amino acid sequence; or a nucleotide sequence having at least 80% identity with the polypeptide, or a nucleotide sequence having at least 85% identity with the polypeptide, or a nucleotide sequence having at least 90% identity with the polypeptide, or a nucleotide sequence having at least 95% identity with the polypeptide; the first conducting domain has an amino acid sequence of SEQ ID NO.8 and a nucleotide sequence of SEQ ID NO.7.

10. A method for preparation of a chimeric antigen receptor or tumor immunotherapy, comprising using the signaling domain as recited in claim 8.
